(19) United States
(12) Patent Application Publication
(10) Pub. No.: US 2008/0046037 A1
(43) Pub. Date: Feb. 21, 2008

(54) Wireless Communication Network for an Implantable Medical Device System

(76) Inventors: Gregory J. Haubrich, Champlin, MN (US); Sarah A. Audet, Shoreview, MN (US); Gerard J. Hill, Champlin, MN (US); Randall L. Knoll, Stillwater, MN (US); Javaid Masoud, Shoreview, MN (US)

Correspondence Address: MEDTRONIC, INC., 710 MEDTRONIC PARKWAY NE, MINNEAPOLIS, MN, 55432-9924, US

(21) Appl. No.: 11/739,283

(22) Filed: Apr. 24, 2007

Related U.S. Application Data
(60) Provisional application No. 60/822,770, filed on Aug. 18, 2006.
(60) Provisional application No. 60/913,394, filed on Apr. 23, 2007.

(51) Int. Cl. A61N 1/02

(52) U.S. Cl. 607/60

(57) ABSTRACT

An implantable medical device system includes, in one embodiment, a first device including a first communication module coupled to a wireless communication network for transmitting data and a second device adapted for implantation in a patient's body including a second communication module coupled to the wireless communication network and adapted to receive data from the first device. The second device may include an equalizer coupled to the second communication module for reducing signal distortion of the data received wirelessly through the patient's body. The second device may convert a received signal to an acoustic or radio-frequency output signal.

### CROSS REFERENCE TO PRIORITY APPLICATION

This application claims priority to provisional application Ser. No. 60/822,770, filed Aug. 18, 2006 and entitled, “Wireless Communication Network for an Implantable Medical Device System” and also claims priority to provisional application Ser. No. 60/913,394, filed Apr. 23, 2007, entitled “Wireless Communication Network for an Implantable Medical Device System”, which is incorporated by reference herein.

### TECHNICAL FIELD

The invention relates generally to implantable medical devices and, in particular, to a communication network for use in implantable medical device systems.

### BACKGROUND

A wide variety of implantable medical devices (IMDs) are available for monitoring physiological conditions and/or delivering therapies. Such devices may include sensors for monitoring physiological signals for diagnostic purposes, monitoring disease progression, or controlling and optimizing therapy delivery. Examples of implantable monitoring devices include hemodynamic monitors, ECG monitors, and glucose monitors. Examples of therapy delivery devices include devices enabled to deliver electrical stimulation pulses such as cardiac pacemakers, implantable cardioverter defibrillators, neurostimulators, and neuromuscular stimulators, and drug delivery devices, such as insulin pumps, morphine pumps, etc.

IMDs are often coupled to medical leads, extending from a housing enclosing the IMD circuitry. The leads carry sensors and/or electrodes and are used to dispose the sensors/electrodes at a targeted monitoring or therapy delivery site while providing electrical connection between the sensor/electrodes and the IMD circuitry. Leadless IMDs have also been described which incorporate electrodes/sensors on or in the housing of the device.

IMD function and overall patient care may be enhanced by including sensors distributed to body locations that are remote from the IMD. However, physical connection of sensors distributed in other body locations to the IMD in order to enable communication of sensed signals to be transferred to the IMD can be cumbersome, highly invasive, or simply not feasible depending on sensor implant location. An acoustic body bus has been disclosed by Funke (U.S. Pat. No. 5,113,859) to allow wireless bidirectional communication through a patient's body. As implantable device technology advances, and the ability to continuously and remotely provide total patient management care expands, there is an apparent need for providing efficient communication between implanted medical devices distributed through a patient's body or regions of a patient's body, as well as with devices located external to a patient's body. Data signals transmitted wirelessly through a patient's body may be subject to considerable dispersion and reflection due to the diversity of body tissue composition and structure encountered as a signal travels through the body between nodes of an implanted medical device communication system.

### BRIEF DESCRIPTION OF THE DRAWINGS

FIG. 1 is a schematic diagram of a wireless communication network implemented in an implantable medical device system.

FIG. 2 is a functional block diagram summarizing functional components included in a networked implantable medical device according to one embodiment of the invention.

FIG. 3 is a block diagram of two networked devices adapted for wireless communication along an intrabody communication pathway.

FIG. 4 is a flow chart of a method for use in an implantable device communication system.

FIG. 5 is a schematic diagram of an implantable medical device communication network including an acoustic/RF gateway node.

FIGS. 6A-6C are schematic diagrams of different implementations of an acoustic/RF gateway node 302 in a communication network.

FIG. 7 is a flow chart relating to a communication method for use in an implantable medical device communication network.

### DETAILED DESCRIPTION

In the following description, references are made to illustrative embodiments for carrying out the invention. It is understood that other embodiments may be utilized without departing from the scope of the invention. For purposes of clarity, the same reference numbers are used in the drawings to identify similar elements. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

The present invention is directed to providing a wireless communications network implemented in an implantable medical device system, wherein the network includes at least one implanted device in communication with a second device, located internally or externally to the patient. The network may be configured having single pathways between networked devices or as a mesh network that allows data to be routed between networked devices through node-to-node routes that can include multiple node “hops” as generally disclosed in U.S. patent application Ser. No. ______, Attorney Docket No. P25563, incorporated herein by reference in its entirety. Embodiments of the invention are not limited to particular network architecture. Among the other types of network architecture which may be used are star, ad hoc, and ALOHA networks. As used herein, the term “node” refers to a device included in a wireless network capable of at least transmitting and/or receiving data on the network and may additionally include other functions as will be described herein. A node can be either an implanted or an external device and is also referred to herein as a “network member”. The wireless network may include multiple implantable devices each functioning as individual network nodes and may include external devices functioning as network nodes as will be further described herein. It is recognized that an overall medical device system implementing a wireless communication network according to various embodiments of the present invention may further include non-networked devices (implantable or external).

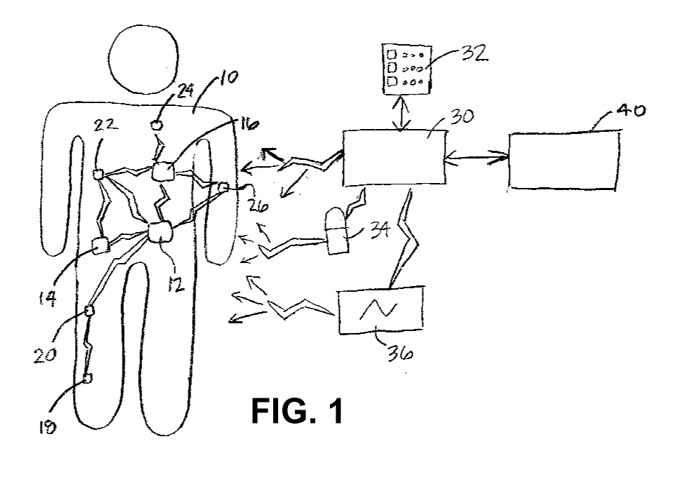

FIG. 1 is a schematic diagram of a wireless communication network implemented in an implantable medical device system. The network includes multiple implantable devices 12 through 26 each functioning as a node (network member). The network may further include external devices functioning as network nodes. Patient 10 is implanted with multiple medical devices 12 through 26 each of which may include physiological sensing capabilities and/or therapy delivery capabilities. As will be further described herein, some of the implanted devices 12 through 26 may be implemented as specialty nodes for performing specific network functions such as data processing, data storage, or communication management functions without providing any physiological sensing or therapy delivery functions.

For example, device 12 may be a therapy delivery device such as a cardiac pacemaker, implantable cardioverter defibrillator, implantable drug pump, or neurostimulators. Device 16 may also be a therapy delivery device serving as a two-way communication node and may further be enabled for performing specialty network management functions such as acting as a network gateway. Device 14 may be embodied as a sensing device for monitoring a physiological condition and also serve as a two-way communication node. Devices 18, 22, 24, and 26 may be embodied as sensing devices for monitoring various physiological conditions and may be implemented as low-power devices operating primarily as transmitting devices with no or limited receiving capabilities. Device 20 may be implemented as a repeater node for relieving the power requirement burden of sensing device 18 for transmitting data from a more remote implant location to other network nodes.

Implantable devices that may be included as network members include any therapy delivery devices, such as those listed above, and any physiological sensing devices such as EGM/ECG sensors, hemodynamic monitors, pressure sensors, blood or tissue chemistry sensors such as oxygen sensors, pH sensors, glucose sensors, potassium or other electrolyte sensors, or sensors for determining various protein or enzyme levels. The wireless network communication system provided by various embodiments of the present invention is not limited to any specific type or combination of implantable medical devices.

The wireless communication network implemented between the implanted devices 24 through 26 may utilize acoustic, ultrasonic and/or radio signal frequency bandwidths. As will be described herein a combination of RF and acoustic or ultrasonic data transmission channels may be implemented to allow simultaneous RF and acoustic/ultrasonic data transmissions. As used herein, “acoustic” signals includes signals in the audible and ultrasonic range.

The wireless network communication system allows a multiplicity of devices to be implanted in a patient as dictated by anatomical, physiological and clinical need, without restraints associated with leads or other hardwire connections through the body for communicating signals and data from one device to another. As such, sensors and/or therapy delivery devices may be implanted in a distributed manner throughout the body according to individual patient need for diagnostic, monitoring, and disease management purposes. Data from the distributed system of implanted sensors and/or therapy delivery devices is reliably and efficiently transmitted between the implanted devices for patient monitoring and therapy delivery functions and may be transmitted to external devices as well for providing patient feedback, remote patient monitoring etc.

The implanted devices 12 through 26 may rely on various power sources including batteries, storage cells such as capacitors or rechargeable batteries, or power harvesting devices relying for example on piezoelectric, thermoelectric or magnetoelectric generation of power. The distributed devices can be provided having minimal power requirements and thus reduced overall size. Implantable devices functioning as network nodes may be miniaturized devices such as small injectable devices, devices implanted using minimally invasive techniques or mini-incisions, or larger devices implanted using a more open approach.

The network may include external devices as shown in FIG. 1 such as a home monitor 30, a handheld device 34, and external monitoring device 36. Reference is made to commonly-assigned U.S. Pat. No. 6,249,703 (Stanton et al.) regarding a handheld device for use with an implantable medical device, hereby incorporated herein by reference in its entirety. The medical device system may further include external devices or systems in wireless or wired communication with external networked devices such as a patient information display 32 for displaying data retrieved from the network to the patient, and a remote patient management system 40. Physiological and device-related data feedback is available to the patient or caregiver via the home monitor 30 and patient information display 32. The home monitor 30, in this illustrative example, includes RF receiver and long range network functionality allowing data received from the implanted network nodes to be accumulated and prioritized for further transmission to the remote patient management system 40 and/or patient information display 32. The patient can respond appropriately to information retrieved from the network and displayed on patient information display 32 in accordance with clinician instructions. A patient may respond, for example, by modifying physical activity, seeking medical attention, altering a drug therapy, or utilizing the handheld device 34 to initiate implanted device functions.

Data can also be made available to clinicians, caregivers, emergency responders, clinical databases, etc. via external or parallel communication networks to enable appropriate and prompt responses to be made to changing patient conditions or disease states. Data acquired by the implantable may be aggregated, for example by a gateway node, and can be further filtered, prioritized or otherwise adjusted in accordance with patient condition and therapy status by a network member to provide clinically meaningful and useful information to a clinician or remote patient management system in a readily-interpretable manner. The home monitor 30 may be coupled to a remote patient monitoring system. Reference is made, for example, to commonly-assigned U.S. Pat. No. 6,599,250 (Webb et al.), U.S. Pat. No. 6,442,433 (Linberg et al.) U.S. Pat. No. 6,622,045 (Snell et al.), U.S. Pat. No. 6,418,346 (Nelson et al.), and U.S. Pat. No. 6,480,745 (Nelson et al.) for general descriptions of network communication systems for use with implantable medical devices for remote patient monitoring and device programming, all of which are hereby incorporated herein by reference in their entirety.

Home monitor 30 and/or a programmer may be used for communicating with one or more of implanted devices 12 through 26 using bidirectional RF telemetry for programming and/or interrogating operations. Reference is made to commonly-assigned U.S. Pat. No. 6,482,154 (Haubrich et al.), hereby incorporated herein by reference in its entirety, for an example of one appropriate long-range telemetry system for use with implantable medical devices. As will be described herein, home monitor 30 may communicate via an RF telemetry link with a gateway node which aggregates acoustical and RF signals received from other implanted devices.

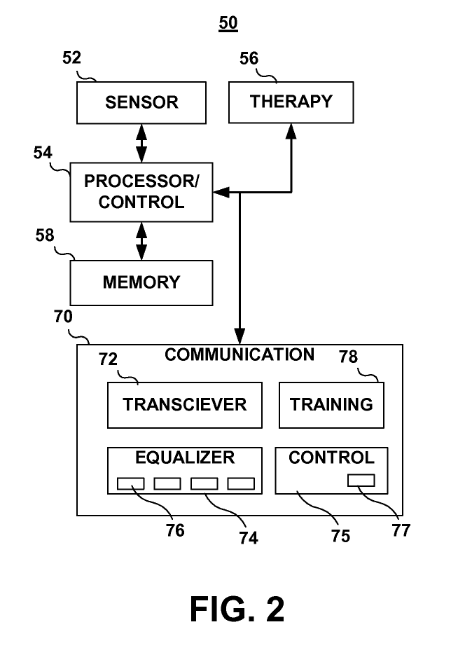

FIG. 2 is a functional block diagram summarizing functional components included in a networked implantable medical device according to one embodiment of the invention. Device 50 generally includes a sensor module 52 for monitoring physiological signals; a therapy delivery module 54 for delivering a therapy in response to the physiological signals according to a programmed operating mode; and a processor/control module 56 and associated memory 58 for controlling device functions. Device 50 further includes a communications module 70 provided with a transceiver 72, an adaptive equalizer 74, a training sequence generator 78, and control circuitry 76. Adaptive equalizer 74 includes multiple adjustable taps 76 that allow optimization of the gain, phase, and delay settings for equalizer 74.

Therapy delivery functions provided by therapy delivery module 54 and physiological monitoring functions provided by sensor module 52 may correspond to the examples provided above. It is recognized that, in some embodiments, device 50 may be provided as a monitoring device without including therapy delivery module 54. Alternatively, device 50 may be a therapy delivery device that does not include sensing capabilities provided by a sensor module 52. Furthermore, device 50 may be a networked device implanted to perform communication functions within an implantable medical device communication system without including either sensing or therapy delivery functions. In some embodiments, sensor module 52 includes or is coupled to a posture and/or activity sensor 53 for sensing changes in body position or changes in patient activity likely to correspond to a change in body position. Changes in body position may alter the signal transmission properties along a particular intrabody communication pathway. As such, detected changes in body position may be used as a trigger for repeating a training session for optimizing adaptive equalizer 74 included in the device communication module 70, as will be further described below.

Communications module 70 is adapted to transmit and receive acoustic intrabody data transmissions. In alternative embodiments, communications module 70 is adapted to transmit and/or receive radio-frequency (RF) data transmissions. In still other embodiments, communications module 70 is adapted to transmit and/or receive both acoustic and RF data transmissions. Data transmitted wirelessly through intrabody connections is subject to distortion and delay due to signal dispersion, reflection, and absorption by the body tissues. Acoustic communication signals are particularly vulnerable to distortion of due to dispersion, reflection, and absorption by the body tissues, however, distortion of RF signals, particularly wide band, ultra wide-band, or impulse RF signals may also occur. Inter-symbol interference occurs as a result of signal delays, limiting the maximum data transfer rate that is used in transmitting acoustical signals within the body. The implementation of adaptive equalizer 74 in communication module 70 allows distortion and delay of transmitted signals to be corrected or compensated for, thereby allowing reliable data transmission to occur at faster data rates.

Equalizer 74 may be implemented digitally, using MOS, CMOS or other integrated circuit technology. Equalizer 74 may be embodied as a digital signal processing block, a shift register with tabs, for example using arithmetic logic units (ALUs), state machine, microprocessor or any other digital circuitry configured to perform the signal equalizing functions described herein. Equalizer 74 is provided with multiple adjustable taps 76 to allow the gain, phase and delay of taps 76 to be adjusted for optimum equalization of received signals. Tap adjustment may be performed by changing multiplication coefficients in respective multiplication circuits in equalizer 74. The outputs of each tap are summed to produce the equalizer output, which is corrected for distortion and delay that may occur during intrabody transmission. Control circuitry 76 may be included in communication module 70 for determining and setting the optimal tap coefficients. Alternatively, processor/control module 56 may execute algorithms for determining optimum equalizer coefficients and provide control signals to equalizer 74 for appropriately setting the tap coefficients. As used herein, “tap settings” and “tap coefficients” are used interchangeably and generally refer to any of the adjustable gain, delay and/or phase of the multiple equalizer taps adjusted to optimize equalization of communication signals. The number of taps and the defined ranges for adjustable gain, delay, and/or phase will be determined according to a particular application and system characteristics, such as expected data rates, data characteristics, communication pathways, etc.

Communications module 70 may further include a training sequence generator 78, which can be implements as a digital state machine or other dedicated digital circuitry, for generating a training sequence to be transmitted to another implanted device for use in determining optimal equalization tap settings. A training sequence may be a pseudo-random noise code or other sequence developed to provide a range of data frequencies, amplitudes and data rates that are expected to be encountered during communication network transmissions. The transmitted training sequence is defined such that it will be representative of the distortion and delay characteristics corresponding to a transmission pathway between the transmitting and receiving devices. The control circuitry 75 includes memory 77 for storing a reference sequence that corresponds to a training sequence generated by another network member to be received by device 50. The training sequence received by device 50 is used by control circuitry 75 for adjusting the taps 76 of equalizer 74. Control circuitry 75 “knows” that the received training sequence should be equal to the stored reference sequence. The control circuitry 76 adjusts the taps 76 until the received training sequence matches the stored reference sequence.

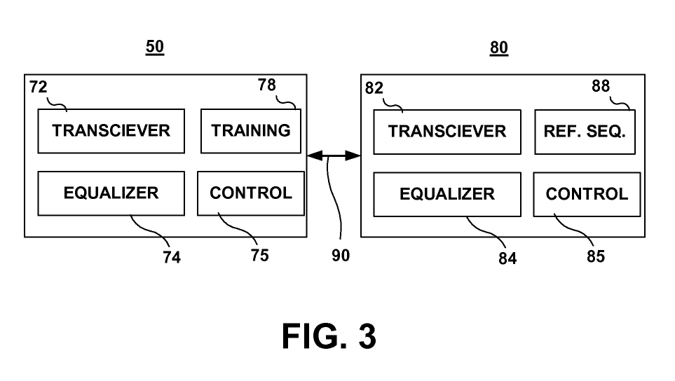

FIG. 3 is a block diagram of two networked devices adapted for wireless communication along an intrabody communication pathway. Device 50 corresponds to the device shown in FIG. 2 and includes communication module as described previously, including transceiver 72, adaptive equalizer 74, control circuitry 75, and training sequence generator 78. Device 80 also includes a communication module including a transceiver 82, adaptive equalizer 84 and control circuitry 85. Device 80 further includes memory 88 for storing a reference sequence. Other functional blocks of device 50 are not shown in FIG. 3 for the sake of simplicity. Likewise, device 80 may include other functional components not shown in FIG. 3.

Device 50 transfers a training sequence generated by training sequence generator 78 to device 80. The training sequence is received by transceiver 82. Control circuitry 85 adjusts the gain, phase and delay of multiple equalizer taps included in equalizer 84 until the received training sequence acceptably matches the reference sequence stored in memory 88. After determining the optimal tap settings, the control signals used for adjusting equalizer 84 to the determined optimal settings in device 80 are transmitted from device 80 to device 50. Control circuitry 75 of device 50 may then use the transmitted control signals for adjusting tap setting for equalizer 74. In this way, the equalizers 74 and 84 of both devices 50 and 80 are optimized by performing one training sequence transmission. This network operation assumes that signals being transmitted between devices 50 and 80 will undergo similar distortion and delay regardless of transmission direction along the intrabody path 90.

Depending on the anatomical characteristics of intrabody path 90, a transmitted signal may be subject to different reflections and dispersions when traveling in one direction than the other. Accordingly, in other embodiments, each of device 50 and device 80 include a training sequence generator and memory for storing a reference sequence such that equalizers 74 and 84 are each optimized during individual training sessions that include transmitting a training sequence and adjusting the tap settings until the received training sequence acceptably matches a reference sequence.

When device 50 is included in a network that includes multiple pathways between multiple devices, a training session may be performed for each of the other devices that device 50 will be communicating with. If device 50 is intended to receive data from multiple devices, a set of tap coefficients for optimal equalization of received signals corresponding to each of the multiple devices may be determined. The appropriate set of tap coefficients would then be provided to equalizer 74 by control circuitry 75 corresponding to the transmitting device from which device 50 is receiving data. Recognition of the transmitting device would be based on time, frequency, or code division multiple access channel plans, identifier code, RFID, or other methods. Alternatively, device 50 may include multiple equalizers each dedicated for equalizing signals received from a specified device or set of devices. Each equalizer would be adjusted to optimal tap setting corresponding to the specified device(s).

In a mesh network application, device 50 may function as a repeater node for transmitting signals between two communicating devices. As such, equalizer 74 may be optimized for receiving signals from the device originating a signal transmission and then transmit the equalized signal on to a final receiving device. The final receiving device may equalize the signal received from device 50 according to previously optimized equalizer tap settings. Alternatively, network nodes used during multiple hops along a mesh network communication route may transmit a signal received from the originating device as is. The final receiving device would be optimized to equalize the final received signal. As such a training sequence may be transmitted from an originating device to one or more intermediate nodes along a multi-hop route to a final receiving device, according to a defined routing scheme and channel plan. Communication module control circuitry in the final receiving device would optimize equalizer tap coefficients such that the final received training sequence matches a stored reference sequence. The optimized tap settings in the final receiving device would correct and compensate for signal distortion and delays occurring along each of the multiple hops.

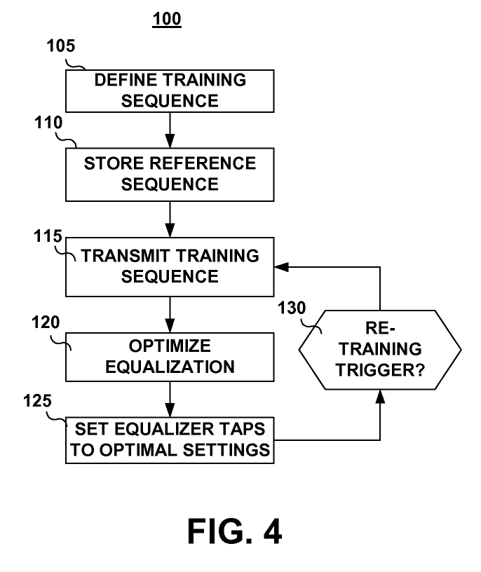

FIG. 4 is a flow chart of a method for use in an implantable device communication system. Method 100 is intended to illustrate the functional operation of the system, and should not be construed as reflective of a specific form of software or hardware necessary to practice the invention. It is believed that the particular form of software/hardware will be determined primarily by the particular system architecture employed in the device and by the particular power capacity and other functional aspects of the device. Providing software to accomplish the present invention in the context of any modern implantable device, given the disclosure herein, is within the abilities of one of skill in the art.

Method 100 relates to a training session used for optimizing an adaptive equalizer in an implanted network node configured to receive acoustic, wideband/ultra-wideband RF, or other communication signals that are subject to distortion and delay along an intrabody communication path. At block 105, a training sequence is defined. As described previously, the training sequence includes a data sequence designed to include the data amplitudes, frequencies, and rates of anticipated data transmissions and thus be representative of the characteristic signal distortions and delays associated with the particular communication path. The training sequence selected will depend on the particular application and network configuration. The training sequence is stored in a transmitting network node corresponding to the communication path for which the training sequence was developed.

At block 110, a reference sequence is stored in a receiving node corresponding to the communication path for which the training sequence was developed. The reference sequence matches the training sequence. At block 115 the training sequence is transmitted from the transmitting node to the receiving node. The receiving node responds to the training sequence by executing an equalization optimization algorithm at block 120. During the equalization optimization, equalizer tap settings or coefficients are automatically adjusted such that the received training sequence, subjected to distortion and delay along the communication path, matches the corresponding stored reference sequence. The optimized tap settings for the corresponding pathway are either stored or applied to the equalizer taps at block 125. Multiple sets of tap settings may be stored for multiple receiving pathways for a given receiving node. As such, method 100 may be repeated for each of the transmitting devices/pathways from which the receiving device will be receiving data communications.

The training sequence may be repeated by returning to block 115. Training sessions may occur on a continuous, periodic or triggered basis. The communication system generally operates in a training mode and a tracking mode. During the training mode, the training sequence is transmitted and the equalizer is optimized to match the received training sequence to a stored reference sequence. During the tracking mode, a node is receiving data transmissions and the adaptive equalizer may be continuously or periodically adjusted. In some embodiments, steps 115 through 125 may be performed at the initiation of every communication session such that the equalizer of the receiving node is adjusted at the beginning of each session. A data transmission may include a data header that includes the training sequence. Depending on the length of the transmission, a training sequence may be provided as a data packet header to allow equalizer optimization during transmission. Continuous or near-continuous adjustment of equalizer settings during the tracking mode allows for correction of data distortion due to even small changes in the communication pathway, e.g. due to minor shifts in patient position. Alternatively, the equalizer may be optimized every nth communication session, or on a scheduled basis, for example every 60 seconds, hourly, daily etc. The frequency of the training sessions may be application specific, depending, for example, on the anticipated frequency of communication sessions and the potential variability in the acoustical properties of the communication pathway. For example, if a patient is sleeping, less frequent changes to the transmission properties of a communication pathway may occur, requiring less frequent equalizer adjustment than during the day when the patient is active and moving about.

In some embodiments, a feedback signal may be used to trigger a training session at block 130. For example, a frequency or number of data errors may trigger the communication system to request a re-training of the adaptive equalizer. In some embodiments, the transmission data rate may be reduced in response to a request for re-training to reduce transmission errors. After the requested training session has been performed, the data rate may be increased again.

It is further contemplated that a position or activity sensor signal indicating a change in patient position may trigger a training session. Changes in patient position, weight, water retention, tissue composition due to disease state, growth, etc., or other anatomical factors may alter the acoustical transmission properties of a communication pathway, warranting a re-optimization of equalization tap settings.

It is further contemplated that certain conditions, such as certain anatomical changes may warrant redefining the training sequence stored at block 105. For example, significant weight gain or the progression of diseased tissue such as tumor growth, tissue swelling, heart dilation or other tissue changes, may alter the characteristic dispersion of acoustical signals along a particular communication pathway. As such, the training sequence and reference sequence may be updated from time to time, and the training session may be repeated as often as needed for maintaining optimal equalization.

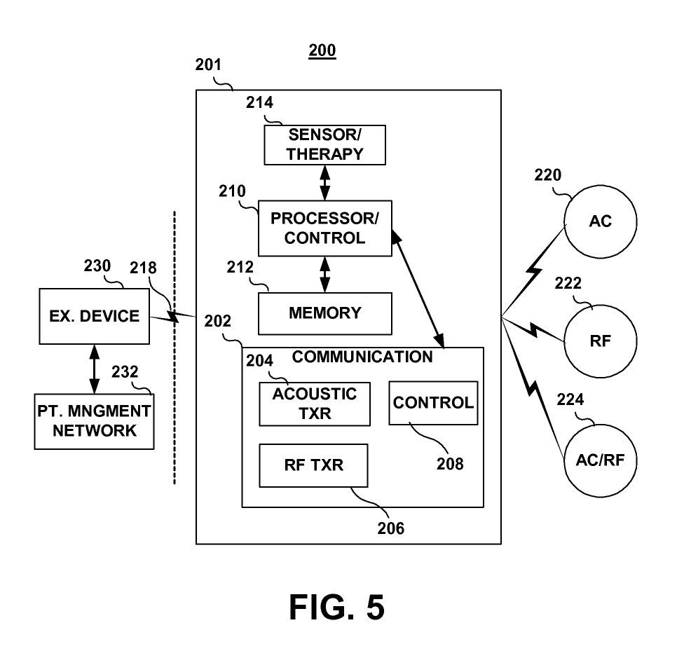

FIG. 5 is a schematic diagram of an implantable medical device communication network including an acoustic/RF gateway node. Acoustic/RF gateway node 201 is provided in network 200 which includes networked devices communicating in the acoustical range and networked devices communicating in the RF range. Accordingly, acoustic/RF gateway node 201 includes a communication module 202 that may include an acoustic signal transceiver 204, an RF signal transceiver 206, and associated control circuitry 208. In some embodiments acoustic/RF gateway node 201 may function as an acoustical receiver and an RF transmitter, without including acoustic transmitting capabilities and/or RF receiving capabilities. Acoustic/RF gateway node 201 may include one or more adaptive equalizers associated with at least acoustic signal transceiver 204. Acoustic/RF gateway node 201 may further include a processor/control module 210 and associated memory 212 and a sensor/therapy delivery module 214.

In one embodiment, one or more RF networked devices 222 transmit data to acoustic/RF gateway node 201 and one or more acoustic networked devices 220 transmit data to acoustic/RF gateway node 201. Acoustic and RF data may be received by acoustic/RF gateway node 201 concurrently. Additionally or alternatively, network 200 may include one or more dual communication devices 224 capable of transmitting acoustic and RF data concurrently to acoustic/RF gateway node 201. Acoustic/RF gateway node 201 receives acoustic and RF data transmissions and concatenates or aggregates the acoustic and RF data. Acoustic/RF gateway node 201 may use received acoustic and RF data in controlling sensing/therapy delivery functions of node 201. Alternatively, acoustic/RF gateway node 201 may convert acoustic data to RF data and transmit the converted data, which may be aggregated with other received RF data, to another implanted device 222 or 224 for use in controlling therapy delivery and/or sensing functions of system 200.

Acoustic/RF gateway node 201 may transmit the aggregated data via an RF telemetry link 218 to an external device 230, such as a programmer or home monitor. As such, acoustic/RF gateway node 201 provides a communication link for transmitting acoustic data acquired by an implanted system to an external communication network node. External device 230 may be coupled to a remote patient management network 232 or other clinician information network such that data received by external device 230 may be made available to clinicians, medical centers or other caregivers in a remote patient management environment.

Acoustic/RF gateway node 201 may be implemented in an implantable device, such as a cardiac stimulation device, neurostimulator, drug delivery device, or physiological monitoring device, or in a specialized communication network node without including monitoring and/or therapy delivery capabilities.

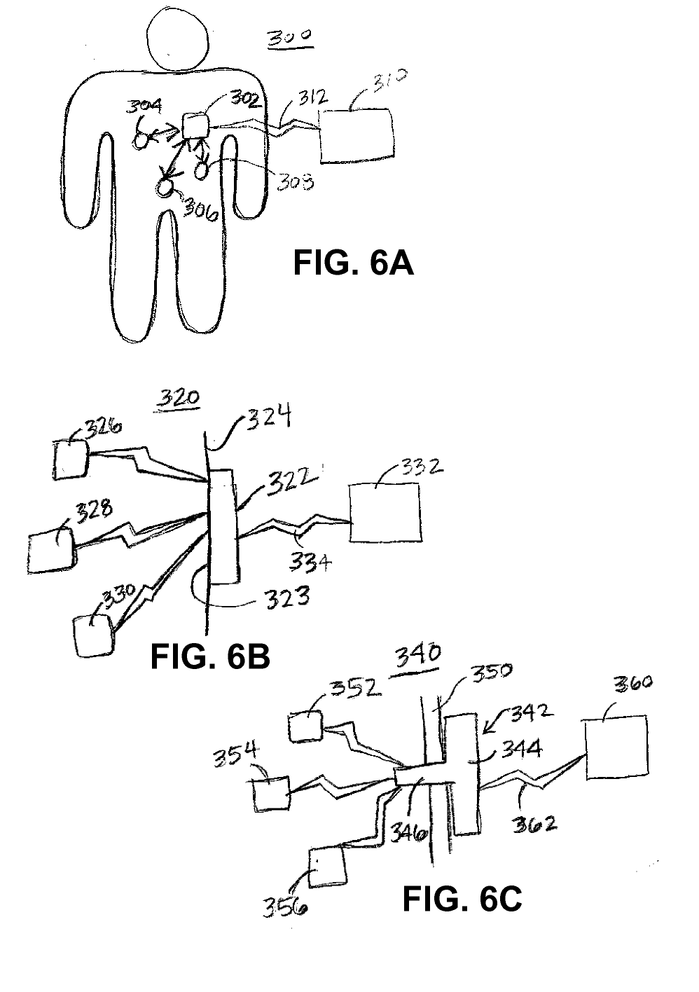

FIG. 6A is a schematic diagram of an implantable medical device communication network 300 including an acoustic/RF gateway node 302 implemented as an implanted device and in communication with one or more implanted devices 304, 306 and 308 in an acoustic communication network. Acoustic/RF gateway node 302 receives acoustic communication signals from each of devices 304, 306 and 308 along respective intrabody communication pathways. Gateway node 302 aggregates the received acoustical data and may further filter, process or prioritize the data. Gateway node 302 converts the aggregated acoustic data to RF data and transmits the RF data to external device 310 via an RF telemetry link 312.

FIG. 6B is a schematic diagram of an implantable medical device communication network including an acoustic/RF gateway node 322 implemented as an external device having a surface 323 adapted for intimate contact with the patient's skin 324 so as to be acoustically coupled with the patient's body. Acoustic/RF gateway node 322 is configured to receive acoustical signals transmitted from one or more devices 326, 328 and 330 implanted within the patient's body. It is contemplated that acoustic/RF gateway node 322 may be implemented in the form of a watch-like device, pager-like device, or other external device that may be comfortably worn by a patient and provide the acoustical coupling needed for receiving acoustical signals being transmitted by implanted devices 326, 328 and 330.

It is further contemplated that in some embodiments, intimate contact with the patient's skin 324 may not be required in order to complete an acoustical communication pathway. Acoustic/RF gateway node 322 may be proximate the patient's skin without making intimate contact and still be adequately acoustically coupled with the implanted devices 326, 328 and 330. For example a layer of clothing may be between the patient's skin 324 and node 322. Gateway node 322 converts aggregates received acoustical data and converts the acoustical data to RF signals which are transmitted to external device 332 via an RF telemetry link 334.

FIG. 6C is a schematic diagram of an acoustic/RF gateway 342 implemented as a transcutaneous device. Acoustic/RF gateway 342 includes an external portion 344 and a transcutaneous portion 346 extending through the patient's skin 350. Transcutaneous portion 346 improves the acoustical coupling between acoustic/RF gateway node 342 and implanted devices 352, 354, and 356. Gateway node 342 receives acoustical data from implanted devices 352, 354, and 356 along respective intrabody acoustical communication paths and converts aggregated acoustical data to RF signals transmitted to external device 360 via RF telemetry link 362.

In any of the networks 300, 320 and 340 shown in FIGS. 6A through 6C, implanted devices may include both acoustical and RF communication links with each other and the acoustic/RF gateway, along concurrent RF and acoustic data transmission. Concurrent acoustic and RF data transmissions may allow fast data transfer between devices, shortening the duration of communication sessions thereby improving data transfer success rates. Acoustic/RF gateway nodes provide a communication path for acoustic data to be transferred to an external device or network. Acoustic/RF gateway nodes may further provide aggregation of acoustic and RF data as described previously.

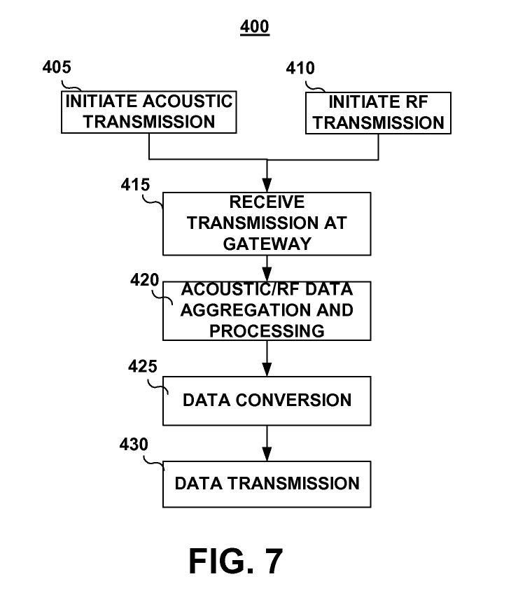

FIG. 7 is a flow chart relating to a communication method for use in an implantable medical device communication network. At block 405 an acoustic data transmission is initiated between an implanted or external network device and an acoustic/RF gateway node. The acoustic/RF gateway may be implanted, external, transcutaneous, or adapted to be worn on the surface of a patient as described above. An acoustic data transmission may be initiated on a scheduled basis, in response to a triggering event, and/or in response to a wake-up signal received by the transmitting device or another specialized network node. The acoustic/RF gateway, or any other network device, may transmit a wake-up signal to another device to initiate an acoustic data transmission.

The acoustic/RF gateway may alternatively receive an RF data transmission from an implanted or external device at block 410. RF data transmissions may occur concurrently or sequentially with acoustic data transmissions. Data transferred may include device programming data, software updates, physiological and/or device related data, which may include patient or physician alert signals, as well as header identifier codes for receiver equalization, security, device identification, data identification or other parametric information such as date, time, etc. Data transmitted is received at the acoustic/RF gateway at block 415. At block 420, the acoustic/RF gateway may aggregate acoustic and RF data received from multiple devices and may perform signal processing and analysis depending on the particular application. The acoustic/RF gateway may use processed or analyzed data for device control operations and/or transmit processed data or analysis results to any other implanted networked device, using acoustic and/or RF communication routes.

The acoustic/RF gateway node may additionally or alternatively convert all acoustic data to RF signals at block 425 for transmission via an RF telemetry link to another implanted or external device at block 430. Alternatively or additionally, the acoustic/RF gateway node may convert all RF data to acoustic data signals at block 425 for transmission via an acoustical link to another implanted, transcutaneous, or surface worn device at block 430.

Thus, an implantable medical device communication system has been presented in the foregoing description with reference to specific embodiments. It is appreciated that various modifications to the referenced embodiments may be made without departing from the scope of the invention as set forth in the following claims.

1. A medical device, comprising:
a communication module wirelessly coupled to a communication network adapted to receive data from a transmitting device along a communication pathway wherein at least a portion of the communication pathway extends through a portion of a patient's body; and
an equalizer coupled to the communication module for reducing signal distortion of the received data along the communication pathway.

2. The device of claim 1 wherein the communication module being further adapted to transmit data.

3. The device of claim 1 wherein the equalizer comprises multiple adjustable taps.

4. The device of claim 3 further comprising a memory for storing a reference sequence for use in automatically adjusting the multiple adjustable taps.

5. The device of claim 3 wherein the multiple adjustable taps each include at least one of an adjustable gain setting, an adjustable phase setting, and an adjustable delay setting.

6. The device of claim 3 further comprising a memory for storing a reference sequence and a control circuit for automatically adjusting the multiple adjustable taps in response to the device receiving a training sequence from the transmitting device wherein the tap adjustments correspond to matching the received training sequence to the stored reference sequence.

7. The device of claim 1 wherein the communication network comprises one of a time division multiple access channel plan, a frequency division multiple access channel plan and a code division multiple access channel plan.

8. The device of claim 1 further comprising one of a sensor module and a therapy delivery module.

9. The device of claim 8 wherein the sensor module comprises one of an electrode, a pressure sensor, flow sensor, a chemical sensor, an acoustical sensor, an ultrasonic sensor, and an accelerometer.

10. The device of claim 8 wherein the therapy delivery module comprises one of an electrical stimulation therapy module and a drug delivery module.

11. The device of claim 1 wherein the equalizer comprises one of a shift register, a digital signal processor, a state machine, and a microprocessor.

12. The device of claim 1 wherein the communication network comprises one of a mesh network, a star network, an ad hoc network and an ALOHA network.

13. The device of claim 1 wherein the communication module being adapted for receiving one of acoustic data signals and radio frequency signals.

14. The device of claim 1 wherein the transmitting device is implanted in the patient's body.

15. An implantable medical device system, comprising:
a first device comprising a first communication module coupled to a wireless communication network for transmitting data; and
a second device adapted for implantation in a patient's body comprising a second communication module coupled to the wireless communication network and adapted to receive data from the first device and further comprising an equalizer coupled to the second communication module for reducing signal dispersion effects on the data received wirelessly through the body.
